(19) United States
(12) Patent Application Publication
(10) Pub. No.: US 2009/0275806 A1
(43) Pub. Date: Nov. 5, 2009

(54) Protable biomedical detective device

(76) Inventors: Tzu-Chiang Wu, Tainan City (TW); Mao-Ji Lin, Sigang Township (TW); Chien-Ho Chuang, Kaohsiung (TW); Tsung-Kai Chuang, Tainan City (TW); Jiann-Hua Wang, Taipei (TW)

Correspondence Address: RABIN & Berdo, PC, 1101 14TH STREET, NW, SUITE 500, WASHINGTON, DC, 20005, US

(21) Appl. No.: 12/114,204

(22) Filed: May 2, 2008

(51) Int. Cl. A61B 5/00

(52) U.S. Cl. 600/300

(57) ABSTRACT

The present invention relates to a portable biomedical detective device comprising a carrier, a detective unit disposed in the carrier, an operating unit for converting biomedical signals of the detection into digitalized results, a display unit shown the digitalized results, and a power supply to drive the detective unit, the operating unit and the display unit. Therefore, biomedical signals on a biomedical cassette can be detected and converted into digital results by the portable biomedical detective device, which is light for carrying easily.

### FIELD OF THE INVENTION

The invention is related to a biomedical detector, more particularly, to a portable biomedical detective device.

### BACKGROUND OF THE INVENTION

The efficiency of the biomedical assay is important for the early diagnosis and therapy of disease. However, it spent a lot of time with conventional biochemistry means in incubating stage and required to combine with different apparatus for the detection process, consequently, the diagnosis result could not be reflected rapidly and accurately. Notwithstanding, it can not help to improve rapid and accurate detection results associated with the biomedical assay without biochip technology developing progressively. While reading and recognizing the biomedical information of a biochip used for assay with a detective device, required to connect with related analyst device or application software. Accordingly, it is difficult and inconvenient to move these heavy old-fashion instruments anywhere for medical services, especially in the faraway area.

### SUMMARY OF THE INVENTION

The main objective of the present invention is to provide a portable biomedical detective device carried easily and has high efficiency to detect and recognize the biomedical information shown on a biomedical cassette.

The portable biomedical detective device, comprising:

a carrier with a hollow portion which is made of a light material and shaped as a box;

at least one detective unit, disposed in the carrier and having at least one socket for inserting a biomedical cassette with biomedical signals;

an operating unit, alternately coupled and decoupled from the carrier, coupled to the detective unit for converting biomedical signals on the biomedical cassette into digitalized results;

a display unit, alternately coupled and decoupled from the carrier, coupled to the operating unit showing the digitalized result; and

a power supply to provide electricity and to drive the detective unit, the operating unit and the display unit.

The detective unit surrounded by a shield material to prevent external light interfering the detection and the portable biomedical detective device further comprises a calibrating element, such as a RFID element, to count the operated number, correct the detective unit and record relative parameters.

Besides, the carrier of the portable biomedical detective device is material of alloy with high-density or plastic resin with high strength to decrease the weight and improve the ability against impact. Besides, because some biomedical detection services are in mountain or somewhere without power supply, the power supply is selected from alternating current (AC) power source, direct current (DC) power source, and/or at least one of removable and rechargeable batteries.

### BRIEF DESCRIPTION OF THE DRAWINGS

The foregoing aspects, as well as many of the attendant advantages and features of this invention will become more apparent by reference to the following detailed description, when taken in conjunction with the accompanying drawings, wherein:

FIG. 1 is a schematic diagram illustrating the open status of the first embodiment in accordance with the present invention.

FIG. 2 is a schematic diagram illustrating the closed status of the first embodiment in accordance with the present invention.

FIG. 3 is a schematic diagram illustrating the second embodiment in accordance with the present invention.

### DESCRIPTION OF THE PREFERRED EMBODIMENT

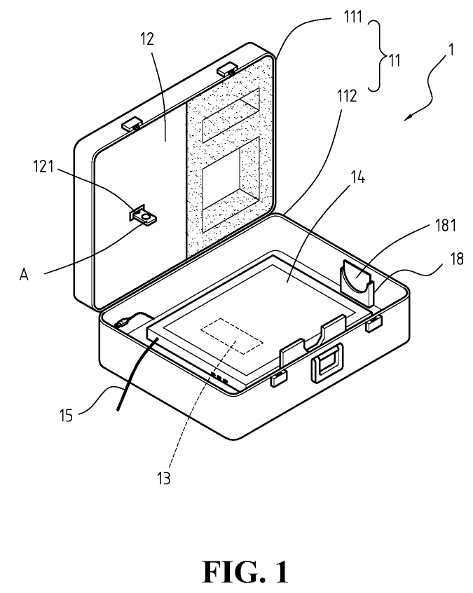

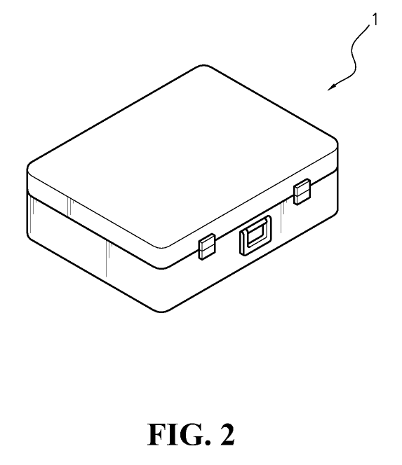

With reference to FIG. 1 and FIG. 2, illustrating the different status of portable biomedical detective device (1) of the present invention. The portable biomedical detective device (1) comprising:

a carrier(11) with a hollow portion which is made of a light material, having an upper layer (111) and a bottom layer (112), when the upper layer (111) and the bottom layer (112) closed, the portable biomedical detective device (1) is shaped as a box or a suitcase for users carrying easily;

at least one detective unit (12), disposed in the carrier (11) and having at least one socket (121) for inserting a biomedical cassette (A) with biomedical signals;

an operating unit(13), alternately coupled and decoupled from the carrier (11) and coupled to the detective unit(12) for converting biomedical signals on the biomedical cassette(A) into digitalized results;

a display unit (14), alternately coupled and decoupled from the carrier (11) and coupled to the operating unit (13) which shows the digitalized biomedical result; and

a power supply (15) in the embodiment utilized a battery to provide electricity and drive the detective unit (12), the operating unit (13) and the display unit (14), wherein the power supply (15) could be selected from AC power source, DC power source, and/or at least one of removable and rechargeable batteries.

The portable biomedical detective device (1) is able to quickly and accurately interpret biomedical information on a biomedical cassette (A), wherein the biomedical cassette (A) is a rapid test trip or array, which the biomedical information on the biomedical cassette (A) is shown in the chromomeric or fluorescent form. After the biomedical cassette (A) with biomedical specimen inserted into the socket (121), biomedical signals of biomedical cassette (A) interpreted by the detective unit (12), and digitalized by the operating unit (13), the biomedical signal with digitalized result is shown on the display unit (14). Therefore, the portable biomedical detective device (1) is possibility to avoid the human error in determining the detection result of the biomedical cassette (A).

In order to avoid the biomedical sample with liquid state flowing, the detective unit (12) set at the upper layer (111) so that the biomedical cassette (A) inserted horizontal to the ground. Besides, the operating unit (13) and the display unit (14) which is a touch panel set at the bottom layer (112) for operating conveniently. Furthermore, the portable biomedical detective device (1) further comprising a calibrating element (18), installed on the bottom layer (112) of the carrier (11), which is a RFID element with a sensor card (181) utilized to correct the detective unit (11), count the operated number, and record relative parameters. The carrier (11) of the portable biomedical detective device (1) is material of alloy with high-density such as the aluminum alloy, titanium alloy, or complex metals. Moreover, the material of the carrier could be a plastic resin with high mechanical intensity such as polyacetal resin (POM) with high strength to decrease the weight and improve the ability against impact.

Furthermore, the detective unit (12) surrounded by a shield material to prevent the detection interfered by external light. And the calibrating element (18) is able to correct the detection result by using a blank to promote the accuracy of the detection.

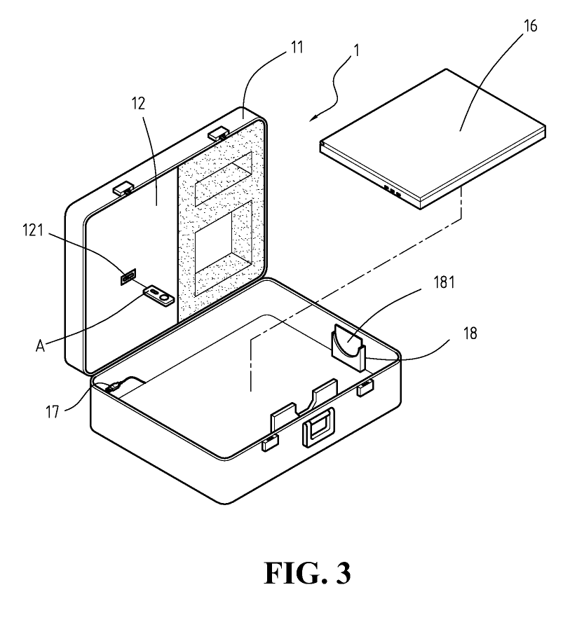

FIG. 3 is a schematic diagram illustrating the second embodiment in accordance with the present invention. The portable biomedical detective device (1) is connected to a notebook (16) through a USB (17). The biomedical signals on the biomedical cassette (A) read by the detective unit (12) and converted to the notebook (16) for recognizing by the analyst software in the notebook (16). In addition, the portable biomedical detective device (1) further comprising a calibrating element (18), which is a RFID element with a sensor card (181), and utilized to correct the detective unit (11), count the operated number, and record relative parameters while detecting.

While the invention has been particularly shown and described with reference to the preferred embodiments thereof, these are, of course, merely examples to help clarify the invention and are not intended to limit the invention. It will be understood by those skilled in the art that various changes, modifications, and alterations in form and details may be made therein without departing from the spirit and scope of the invention, as set forth in the following claims.

What is claimed is:

1. A portable biomedical detective device, comprising:
a carrier with a hollow portion which made of a light material and shaped as a box;
at least one detective unit, disposed in the carrier and having at least one socket for inserting a biomedical cassette;
an operating unit, alternately coupled and decoupled from the carrier and coupled to the detective unit;
a display unit, alternately coupled and decoupled from the carrier and coupled to the operating unit; and
a power supply to provide electricity and to drive the detective unit, the operating unit and the display unit.

2. The portable biomedical detective device as claimed in claim 1, wherein the material of the carrier is an alloy with high density.

3. The portable biomedical detective device as claimed in claim 1, wherein the material of the carrier is a plastic resin.

4. The portable biomedical detective device as claimed in claim 1, wherein the detective unit surrounded by a shield material.

5. The portable biomedical detective device as claimed in claim 1, wherein the portable biomedical detective device further comprising a calibrating element.

6. The portable biomedical detective device as claimed in claim 1, wherein the power supply is current (AC) power source, direct current (DC) power source, and/or at least one of removable and rechargeable batteries.

7. The portable biomedical detective device as claimed in claim 2, wherein the alloy is titanium alloy, aluminum alloy or complex metal.

8. The portable biomedical detective device as claimed in claim 3, wherein the plastic resin is polyacetal resin (POM).

9. The portable biomedical detective device as claimed in claim 4, wherein the shield material is glass fiber or acrylic fiber.

10. The portable biomedical detective device as claimed in claim 5, wherein the calibrating element is a RFID element.
